(19) United States
(12) Patent Application Publication
(10) Pub. No.: US 2020/0370040 A1
(43) Pub. Date: Nov. 26, 2020

(54) TREATMENT FOR PARKINSONIAN PATIENTS WITH MUTATIONS IN THE LRRK2 GENE

(71) Applicant: Ramot at Tel-Aviv University Ltd., Tel-Aviv (IL)

(72) Inventors: Daniel OFFEN, Tel-Aviv (IL); Roy RABINOWITZ, Tel-Aviv (IL)

(73) Assignee: Ramot at Tel-Aviv University Ltd., Tel-Aviv (IL)

(21) Appl. No.: 16/769,260

(22) PCT Filed: Dec. 6, 2018
(86) PCT No.: PCT/IL2018/051336

§ 371 Date: Jun. 3, 2020

Related U.S. Application Data
(60) Provisional application No. 62/595,602, filed on Dec. 7, 2017.

(51) Int. Cl. C12N 15/11; A61K 38/46; C12N 9/22

(52) U.S. Cl. CPC C12N 15/11; A61K 38/465; C12N 2800/80; C12N 2310/20; C12N 9/22

(57) ABSTRACT

A method of treating Parkinson's Disease (PD) characterized by the presence of a mutant allele of leucine-rich repeat kinase 2 (LRRK2) gene in a subject is disclosed. The method comprises administering to the subject a CRISPR-Cas system guide RNA (gRNA) which specifically binds to the mutant allele of said leucine-rich repeat kinase 2 (LRRK2) gene and a CRISPR endonuclease, thereby treating the Parkinson's Disease (PD).

### FIELD AND BACKGROUND OF THE INVENTION

The present invention, in some embodiments thereof, relates to a method of treating Parkinson's disease.

Parkinson's disease (PD) is a neurodegenerative disorder that mainly affects the motor system of the central nervous system (CNS). It is thought to be more common in the elderly, but factors such as genetic factors may cause early onset of the disease. Current estimations suggest that there are over 10 million people worldwide that live with PD. G2019S, a genetic variation (SNP) in the leucine-rich repeat kinase 2 (LRRK2) gene is highly associated with PD, both familial and sporadic. The G2019S mutation (G6055A transition) prevalence in PD patients is about 3-7% in familial PD and 1-2% in sporadic PD. Interestingly, in some populations the prevalence of this variation appears to be as high as 28% in Ashkenazi Jews and 42% in North Africa. A person who carries the G2019S mutation has a high probability of developing PD (28% at age 59 and 74% at age 79). As proven by animal and cell models, LRRK2 mutations affect vesicular trafficking, autophagy, protein synthesis, and cytoskeletal function. Moreover, LRRK2 mutations are all known contribute to degeneration and death of dopamine neurons. Current studies underline the importance of developing LRRK2 inhibitors due to the established link between LRRK2 activity and toxicity.

### SUMMARY OF THE INVENTION

According to an aspect of some embodiments of the present invention, there is provided a method of treating Parkinson's Disease (PD) characterized by the presence of a mutant allele of leucine-rich repeat kinase 2 (LRRK2) gene in a subject comprising administering to the subject a CRISPR-Cas system guide RNA (gRNA) which specifically binds to the mutant allele of the leucine-rich repeat kinase 2 (LRRK2) gene and a CRISPR endonuclease, thereby treating the Parkinson's Disease (PD).

According to embodiments of the present invention, the mutant allele of leucine-rich repeat kinase 2 (LRRK2) gene comprises the G2019S mutation.

According to embodiments of the present invention, the CRISPR endonuclease is Clustered Regularly interspaced Short Palindromic Repeats from Prevotella and Francisella 1 (Cpf1).

According to embodiments of the present invention, the subject is homozygous for a mutation in the LRRK2 gene.

According to embodiments of the present invention, the subject is heterozygous for a mutation in the LRRK2 gene.

According to embodiments of the present invention, a Protospacer adjacent motif (PAM) sequence utilized by the gRNA comprises the G2019S mutation.

According to embodiments of the present invention, the gRNA comprises a nucleic acid sequence as set forth in SEQ ID NO: 3.

According to embodiments of the present invention, the gRNA comprises a nucleic acid sequence selected from the group consisting of SEQ ID NOs: 3-7 and 11-21.

According to embodiments of the present invention, the gRNA comprises a nucleic acid sequence as set forth in SEQ ID NO: 3 or 4.

According to an aspect of some embodiments of the present invention, there is provided a gRNA, which specifically binds to a mutant allele of LRRK2.

According to embodiments of the present invention, the mutant allele of LRRK2 comprises the G2019S mutation.

According to embodiments of the present invention, a Protospacer adjacent motif (PAM) sequence utilized by the gRNA comprises the G2019S mutation.

According to embodiments of the present invention, the gRNA comprises a nucleic acid sequence as set forth in SEQ ID NO: 3.

According to embodiments of the present invention, the gRNA comprises a nucleic acid sequence selected from the group consisting of SEQ ID NOs: 4-7 and 3.

According to embodiments of the present invention, the gRNA comprises a nucleic acid sequence as set forth in SEQ ID NO: 3 or 4.

According to embodiments of the present invention, the gRNA comprises a nucleic acid sequence selected from the group consisting of SEQ ID NOs: 3-7 and 11-21.

According to an aspect of some embodiments of the present invention, there is provided an article of manufacture comprising the gRNA of any one of claims 10-16 and a CRISPR endonuclease.

According to embodiments of the present invention, the CRISPR endonuclease is Clustered Regularly Interspaced Short Palindromic Repeats from Prevotella and Francisella 1 (Cpf1).

According to embodiments of the present invention, the article of manufacture is for use in treating Parkinson's Disease (PD).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 provides a partial sequence of wild type LRRK2 (SEQ ID NO: 1); G2019S mutant LRRK2 (SEQ ID NO: 2) and an exemplary gRNA sequence (SEQ ID NO: 3) that is capable of hybridizing to the mutant sequence.

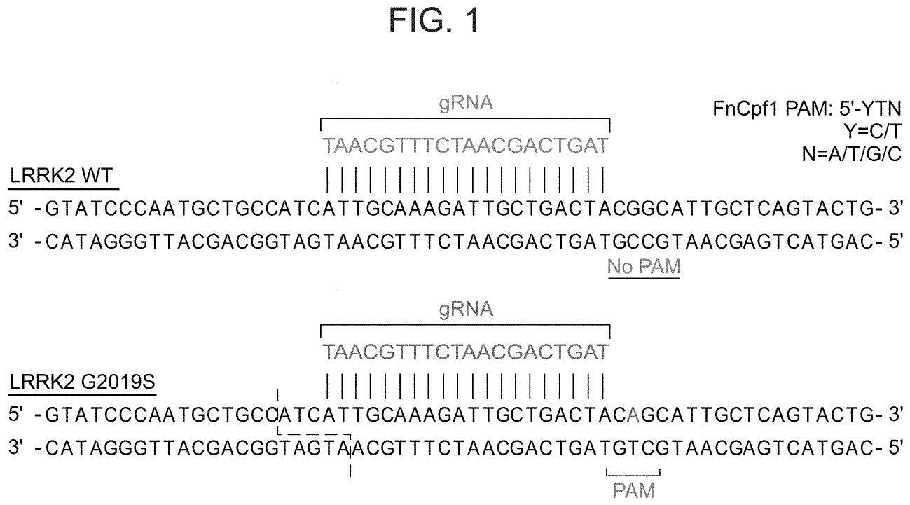

FIG. 2 provides the sequence of wild type LRRK2 (SEQ ID NO: 1); G2019S mutant LRRK2 (SEQ ID NO: 2) and an exemplary gRNA sequence (SEQ ID NO: 4) that is capable of hybridizing to the mutant sequence.

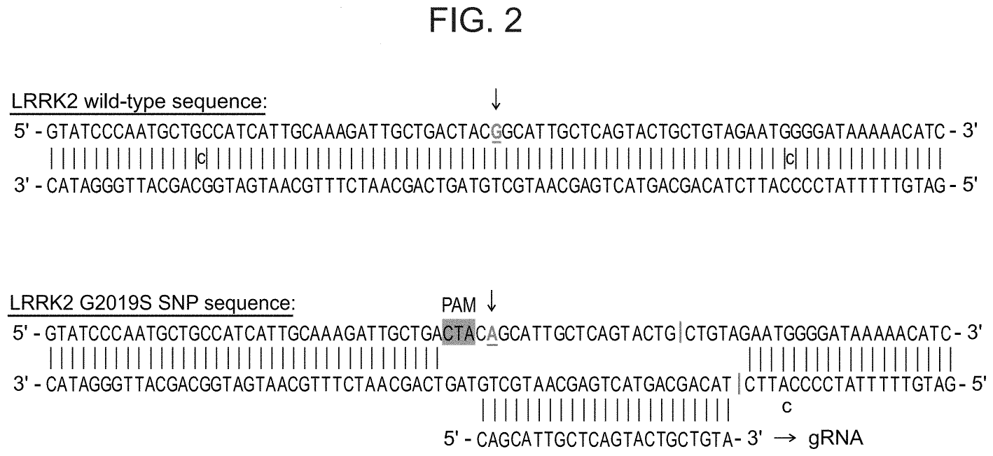

FIG. 3 provides the sequence of wild type LRRK2 (SEQ ID NO: 1); G2019S mutant LRRK2 (SEQ ID NO: 2) and an exemplary gRNA sequence (SEQ ID NO: 5) that is capable of hybridizing to the mutant sequence.

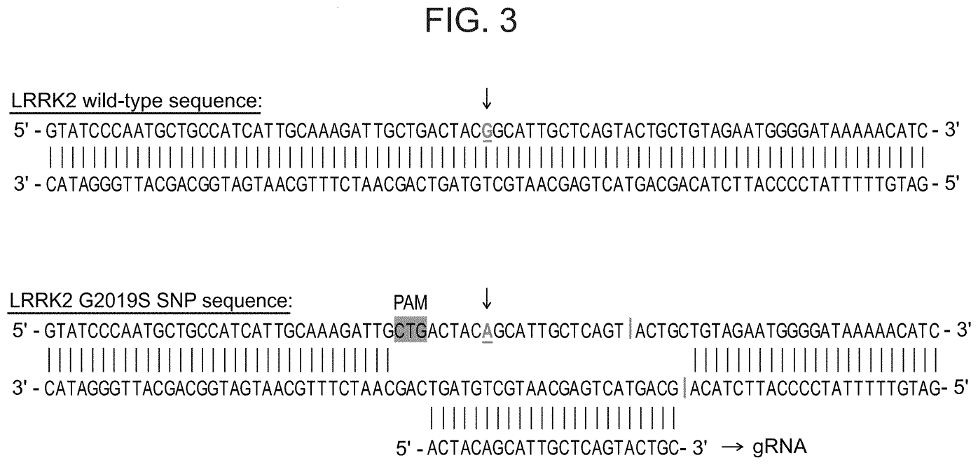

FIG. 4 provides the sequence of wild type LRRK2 (SEQ ID NO: 1); G2019S mutant LRRK2 (SEQ ID NO: 2) and an exemplary gRNA sequence (SEQ ID NO: 6) that is capable of hybridizing to the mutant sequence.

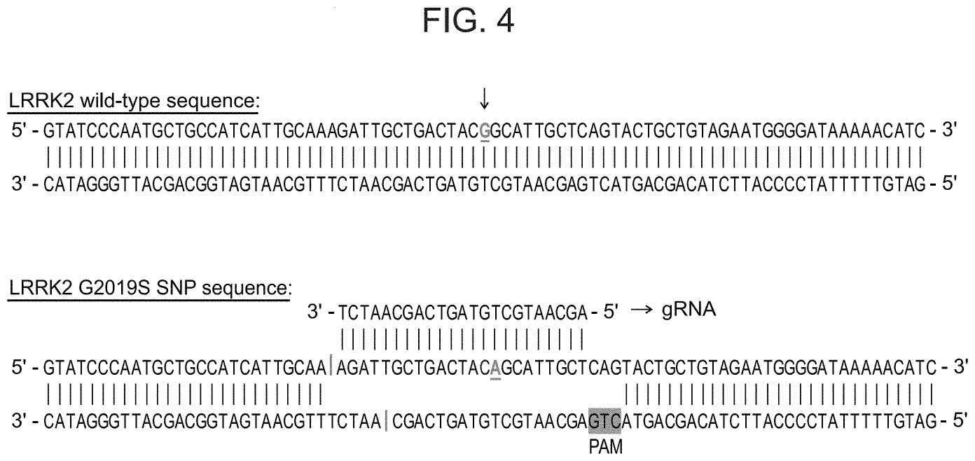

FIG. 5 provides the sequence of wild type LRRK2 (SEQ ID NO: 1); G2019S mutant LRRK2 (SEQ ID NO: 2) and an exemplary gRNA sequence (SEQ ID NO: 7) that is capable of hybridizing to the mutant sequence.

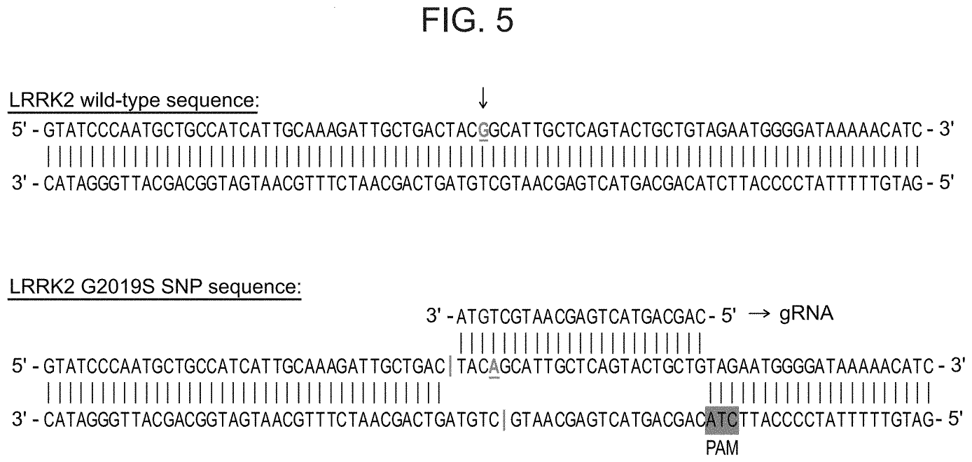

### DESCRIPTION OF SPECIFIC EMBODIMENTS OF THE INVENTION

The present invention, in some embodiments thereof, relates to a method of treating Parkinson's disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The clustered regularly interspaced short palindromic repeat (CRISPR) system enables precise genome editing mediated by a single-guide RNA (sgRNA) that guides the CRISPR associated (Cas) protein to the target DNA in the genome. Cas9, the catalytic unit of the CRISPR system, generates a double-strand break (DSB) in the DNA in the presence of a DNA:sgRNA match and a protospacer-adjacent motif (PAM) in immediate proximity to the target DNA. The diversity of Cas proteins, lies in the PAM sequence recognized thereby, cleavage pattern and position, size, activity in mammalian cells, off-targets and substrate (DNA or RNA). The standard Cas protein has been modified to broaden its applications to base-editing, transcription repression and activation, epigenomic modifications, visualization of genomic loci and DNA nicking (single-strand cleavage).

Previous studies have shown that targeting an allele caused by a SNP by choosing a gRNA sequence containing the variated nucleotide is not a clinical option, since it results in a non-specific knockdown of both the mutant alleles and the wild-type allele. Both the wild-type and the mutant allele are down-regulated to the same degree [Capon, S. J. et al. Biol. Open 6, 125-131 (2017); Christie, K. A. et al. Sci. Rep. 7, 16174 (2017)].

The present inventors have now conceived of a SNP-derived PAM approach, which overcomes this potential limitation. This method dramatically increases the specificity of targeting the mutant allele alone by choosing a PAM sequence that is present only at the mutant sequence i.e. the mutant SNP generates the PAM sequence.

Thus, according to one aspect of the present invention there is provided a method of treating Parkinson's Disease (PD) characterized by the presence of a mutant allele of leucine-rich repeat kinase 2 (LRRK2) gene in a subject comprising administering to the subject a CRISPR-Cas system guide RNA (gRNA) which specifically binds to the mutant allele of the leucine-rich repeat kinase 2 (LRRK2) gene and a CRISPR endonuclease, thereby treating the Parkinson's Disease (PD).

The term “LRRK2” refers to the human gene (Gene ID: 120892) which encodes a protein having a Uniprot No. Q55007.

The DNA sequence of the wild-type gene is provided in SEQ ID NO: 8.

An exemplary amino acid sequence of wild-type human LRRK2 is provided in SEQ ID NO: 9.

It will be appreciated that the gRNA and CRISPR endonuclease disclosed herein are also expected to be useful in the treatment of other diseases, which are associated with LRRK2 mutations. For example, studies show an increased risk of non-skin cancer in LRRK2 Gly2019Ser mutation carriers and especially for renal and lung cancer [see for example Mov. Disorder, 25, 2536-2541, 2010].

The agents of the present invention may be used to treat a subject having any stage of Parkinson's disease, from early stages to the very late stages of the disease.

Preferably, the subject has been diagnosed with Parkinson's disease and has been confirmed to be carrying a LRRK2 mutation.

Methods for analyzing for the presence of an LRRK2 mutation are known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

In one embodiment, the method comprises analyzing for the presence of an LRRK2 mutation in a subject known to have Parkinson's disease or suspected of having Parkinson's disease, and then if the result is positive, recommending and/or providing treatment with the DNA modifying agents disclosed herein.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to one embodiment, the mutant allele of leucine-rich repeat kinase 2 (LRRK2) gene comprises the G2019S mutation.

According to a particular embodiment, the PAM sequence recognized by the gRNA of the present invention comprises the G2019S mutation.

In one embodiment, the alteration in the LRRK2 gene is situated on one allele of the gene. According to other specific embodiments, alteration of a LRRK2 gene comprises both alleles of the gene. In such instances the e.g. LRRK2 may be in a homozygous form or in a heterozygous form. According to this embodiment, homozygosity is a condition where both alleles at the e.g. LRRK2 locus are characterized by the same nucleotide sequence. Heterozygosity refers to different conditions of the gene at the e.g. LRRK2 locus.

Methods of introducing nucleic acid alterations to a gene of interest (e.g. the LRRK2 gene) are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG (SEQ ID NO: 10) family, the GIY-YIG family, the His-Cys box family and the HNH family. These families are characterized by structural motifs, which affect catalytic activity and recognition sequence. For instance, members of the LAGLIDADG (SEQ ID NO: 10) family are characterized by having either one or two copies of the conserved LAGLIDADG (SEQ ID NO: 10) motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme, which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

In one embodiment, the gRNA has a sequence as set forth in SEQ ID NO: 3 and optionally the CRISPR endonuclease enzyme is Cpf1.

In another embodiment, the gRNA has as sequence as set forth in SEQ ID NO: 4, and optionally the CRISPR endonuclease enzyme is Cpf1.

According to still another embodiment, the gRNA has a sequence as set forth in SEQ ID NOs: 5-7 and optionally the CRISPR endonuclease enzyme is Cpf1.

According to still another embodiment, the gRNA has a sequence as set forth in SEQ ID NOs: 11-21. Particular exemplary CRISPR endonuclease enzymes that can be used for each of these gRNAS are set forth in Table 1, herein below.

As mentioned, as well as the gRNA, the CRISPR system utilizes an endonuclease enzyme.

Preferably, the codons encoding the CRISPR endonuclease enzymes are “optimized” codons, i.e., the codons are those that appear frequently in, e.g., highly expressed genes in humans, instead of those codons that are frequently used by, for example, in bacteria. Such codon usage provides for efficient expression of the protein in human cells. Codon usage patterns are known in the literature for highly expressed genes of many species (e.g., Nakamura et al, 1996; Wang et al, 1998; McEwan et al. 1998).

In one embodiment, the CRISPR endonuclease enzyme is Clustered Regularly Interspaced Short Palindromic Repeats from Prevotella and Francisella 1 (Cpf1).

An exemplary DNA sequence of Cpf1 is set forth in SEQ ID NO: 40.

An exemplary amino acid sequence of Cpf1 is set forth in SEQ ID NO: 41.

Preferably, the DNA encodes for a CRISPR endonuclease enzyme having an amino acid sequence at least 90%, 91%, 92%, 93%, 94%, 95%, 96%, 97%, 98%, 99% or 100% identical to the sequence as set forth in 41.

Preferably, the DNA encodes for a CRISPR endonuclease enzyme having an amino acid sequence at least 90%, 91%, 92%, 93%, 94%, 95%, 96%, 97%, 98%, 99% or 100% homologous to the sequence as set forth in 41.

The Cpf1 locus contains a mixed alpha/beta domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 doesn't need the tracrRNA, therefore, only crRNA is required. This benefits genome editing because Cpf1 is not only smaller than Cas9, but also it has a smaller sgRNA molecule (approximately half as many nucleotides as Cas9).

The Cpf1-crRNA complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

In another embodiment, the CRISPR endonuclease enzyme is Cas9.

Cas9 requires two RNA molecules to cut DNA while Cpf1 needs one. Cas9 cuts both strands in a DNA molecule at the same position, leaving behind blunt ends. Cpf1 leaves one strand longer than the other, creating sticky ends. The sticky ends aid in the incorporation of new sequences of DNA, making Cpf1 more efficient at gene introductions than Cas9. Cpf1 lacks tracrRNA, utilizes a T-rich PAM and cleaves DNA via a staggered DNA DSB.

Other examples of gRNAs that can be used to target (and down-regulate) LRRK2 in an allele specific manner are provided in Table 1, listed together with the potential endonuclease enzyme that can be used.

TABLE 1
LRRK2 mutations

| | | | |
|---|---|---|---|
| SNP: | rs33939927 | C to A | missense |

WT sequence: GTTTTGTGTCTTTCCCTCCAGGCTCGCGCTTCTTCTTCCCCTGTGATTC
SEQ ID NO: 22
variant sequence:
GTTTTGTGTCTTTCCCTCCAGGCTAGCGCTTCTTCTTCCCCTGTGATTC
SEQ ID NO: 23

| Cas | FnCpf1 | gRNA | GCCTGGAGGGAAAGACACAA - |
|---|---|---|---|
| type: | | sequence: | SEQ ID NO: 11 |
| SNP: | rs34594498 | C to T | missense |

WT sequence:
CAAAGGAAGTTTTCCAGGCATCTGCGAATGCATTGTCAACTCTCTTAGA
SEQ ID NO: 24
variant sequence:
CAAAGGAAGTTTTCCAGGCATCTGTGAATGCATTGTCAACTCTCTTAGA
SEQ ID NO: 25

| Cas | SpCas9 (from Streptococcus | gRNA | AAAGGAAGTTTTCCAGGCAT - |
|---|---|---|---|
| type: | pasteurianus) | sequence: | SEQ ID NO: 12 |
| SNP: | rs34637584 | G to A | missense |

WT sequence:
ATCATTGCAAAGATTGCTGACTACGGCATTGCTCAGTACTGCTGTAGAA
SEQ ID NO: 26
variant sequence:
ATCATTGCAAAGATTGCTGACTACAGCATTGCTCAGTACTGCTGTAGAA
SEQ ID NO: 27

| Cas | FnCpf1 | gRNA | TAGTCAGCAATCTTTGCAAT - |
|---|---|---|---|
| type: | | sequence: | SEQ ID NO: 13 |
| SNP: | rs34805604 | A to G | missense |

WT sequence:
GTGACTAGAAATAAAATATCAGGGATATGCTCCCCCTTGAGACTGAAGG
SEQ ID NO: 28
variant sequence:
GTGACTAGAAATAAAATATCAGGGGTATGCTCCCCCTTGAGACTGAAGG
SEQ ID NO: 29

| Cas | SpCas9 | gRNA | GTGACTAGAAATAAAATATC - |
|---|---|---|---|
| type: | | sequence: | SEQ ID NO: 14 |
| Cas | xCas9 | gRNA | TGACTAGAAATAAAATATCA - |
| type: | | sequence: | SEQ ID NO: 15 |
| SNP: | rs34995376 | G to A | missense |

WT sequence: TTTTGTGTCTTTCCCTCCAGGCTCGCGCTTCTTCTTCCCCTGTGATTCT
SEQ ID NO: 30
variant sequence:
TTTTGTGTCTTTCCCTCCAGGCTCACGCTTCTTCTTCCCCTGTGATTCT
SEQ ID NO: 31

| Cas | SpCas9 (from Streptococcus | gRNA | GAATCACAGGGGAAGAAGAA - |
|---|---|---|---|
| type: | pasteurianus) | sequence: | SEQ ID NO: 16 |
| SNP: | rs35808389 | A to G | synonymous-codon |

WT sequence:
AGAGAAACTGGAGCAGCTCATTTTAGAAGGGTAAGAAAGAGCTCATTAA
SEQ ID NO: 32
variant sequence:
AGAGAAACTGGAGCAGCTCATTTTGGAAGGGTAAGAAAGAGCTCATTAA
SEQ ID NO: 33

| Cas | SpCas9 | gRNA | GAAACTGGAGCAGCTCATTT - |
|---|---|---|---|
| type: | | sequence: | SEQ ID NO: 17 |
| SNP: | rs74163686 | A to C | missense |

WT sequence:
GATGCCATGAAGCCTTGGCTCTTCAATATAAAGGTGATTTGTTCTGATC
SEQ ID NO: 34
variant sequence:
GATGCCATGAAGCCTTGGCTCTTCCATATAAAGGTGATTTGTTCTGATC
SEQ ID NO: 35

| Cas | SpCas9 | gRNA | CAGAACAAATCACCTTTATA - |
|---|---|---|---|
| type: | | sequence: | SEQ ID NO: 18 |
| SNP: | rs281865052 | A to G | missense |

WT sequence:
TTGGCTGACCTGCCTAGAAATATTATGTTGAATAATGATGAGTTGGAAT
SEQ ID NO: 36
variant sequence:
TTGGCTGACCTGCCTAGAAATATTGTGTTGAATAATGATGAGTTGGAAT
SEQ ID NO: 37

| Cas | xCas9 | gRNA | TGTGTTGAATAATGATGAGT - |
|---|---|---|---|
| type: | | sequence: | SEQ ID NO: 19 |
| SNP: | rs281865054 | G to A | missense |

WT sequence:
AGAAATATTATGTTGAATAATGATGAGTTGGAATTTGAACAAGCTCCAG
SEQ ID NO: 38
variant sequence:
AGAAATATTATGTTGAATAATGATAAGTTGGAATTTGAACAAGCTCCAG
SEQ ID NO: 39

| | | | |
|---|---|---|---|
| Cas | CjCas9 | gRNA | GAATAATGATAAGTTGGAAT - |
| type: | | sequence: | SEQ ID NO: 20 |
| Cas | FnCpf1 | gRNA | ATCATTATTCAACATAATAT - |
| type: | | sequence: | SEQ ID NO: 21 |

In order to use the CRISPR system, both gRNA and CRISPR endonuclease should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence, which can move around to different positions within the genome of a single cell. In the process, the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors, which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

The DNA modifying agents of this aspect of the present invention may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the DNA modifying agents described herein (e.g. CRISPR-Cas system guide RNA and/or CRISPR endonuclease) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

The DNA modifying agents described herein are administered such that they are capable of inhibiting (e.g. downregulating) LRRK2 in the brain of the subject.

In one embodiment, the administration is such that the agents reach the striatum and/or substantia nigra.

In another embodiment, the administration is via systemic CNS transduction e.g. using viral vectors such as AAV9.

According to a specific embodiment, the agents are administered intrathecally (e.g. through a catheter into the CNS).

According to another embodiment, the agents are administered systemically, e.g. intravenously.

According to still another embodiment, the agents are administered intranasally.

Alternately, one may administer the pharmaceutical composition in a local rather than systemic manner, for example, via injection of the pharmaceutical composition directly into the brain of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations, which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions that can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The agents of the present invention may be comprised in particles (e.g. exosomes, microvesicles, nanvesicles, membrane particles, membrane vesicles, ectosomes and exovesicles). In other embodiments, the agents of the present invention may be comprised in synthetic particles (e.g. liposomes). The particles may be administered in any of the above mentioned ways including for example intranasal administration.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (CRISPR endonuclease and/or gRNA) effective to prevent, alleviate or ameliorate symptoms of a disorder (e.g., Parkinson's Disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer, as further detailed below. Such information can be used to more accurately determine useful doses in humans.

Toxicity and therapeutic efficacy of the active ingredients described herein can be determined by standard pharmaceutical procedures in vitro, in cell cultures or experimental animals, as further detailed below. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to ensure blood or tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

As mentioned, various animal models may be used to test the efficacy of the agent of the present invention—e.g. using C57BL/6J-Tg(LRRK2*G2019S)2AMjff/J transgenic mice from Jackson Laboratory or C57BL/6-Lrrk2^{tm4.1Arte mice} from Taconic.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating Parkinson's Disease (PD) characterized by the presence of a mutant allele of leucine-rich repeat kinase 2 (LRRK2) gene in a subject comprising administering to the subject a CRISPR-Cas system guide RNA (gRNA) which specifically binds to the mutant allele of said leucine-rich repeat kinase 2 (LRRK2) gene and a CRISPR endonuclease, thereby treating the Parkinson's Disease (PD).

2. The method of claim 1, wherein said mutant allele of leucine-rich repeat kinase 2 (LRRK2) gene comprises the G2019S mutation.

3. The method of claim 1, wherein said CRISPR endonuclease is Clustered Regularly Interspaced Short Palindromic Repeats from Prevotella and Francisella 1 (Cpf1).

4. The method of claim 1, wherein the subject is homozygous for a mutation in the LRRK2 gene.

5. The method of claim 1, wherein the subject is heterozygous for a mutation in the LRRK2 gene.

6. The method of claim 1, wherein a Protospacer adjacent motif (PAM) sequence utilized by said gRNA comprises said G2019S mutation.

7. The method of claim 6, wherein said gRNA comprises a nucleic acid sequence as set forth in SEQ ID NO: 3.

8. The method of claim 1, wherein said gRNA comprises a nucleic acid sequence selected from the group consisting of SEQ ID NOs: 3-7 and 11-21.

9. The method of claim 8, wherein said gRNA comprises a nucleic acid sequence as set forth in SEQ ID NO: 3 or 4.

10. A gRNA which specifically binds to a mutant allele of LRRK2.

11. The gRNA of claim 10, wherein said mutant allele of LRRK2 comprises the G2019S mutation.

12. The gRNA of claim 11, wherein a Protospacer adjacent motif (PAM) sequence utilized by said gRNA comprises said G2019S mutation.

13. The gRNA of claim 12, wherein said gRNA comprises a nucleic acid sequence as set forth in SEQ ID NO: 3.

14. The gRNA of claim 10, wherein said gRNA comprises a nucleic acid sequence selected from the group consisting of SEQ ID NOs: 4-7 and 3.

15. The gRNA of claim 14, wherein said gRNA comprises a nucleic acid sequence as set forth in SEQ ID NO: 3 or 4.

16. The gRNA of claim 10, comprising a nucleic acid sequence selected from the group consisting of SEQ ID NOs: 3-7 and 11-21.

17. An article of manufacture comprising the gRNA of claim 10 and a CRISPR endonuclease.

18. The article of manufacture of claim 17, wherein said CRISPR endonuclease is Clustered Regularly Interspaced Short Palindromic Repeats from Prevotella and Francisella 1 (Cpf1).

19. (canceled)


SEQUENCE LISTING (41 sequences)

SEQ ID NO: 1 - DNA, 57 bp, Artificial sequence
  partial sequence of WT LRRK2
gtatcccaatgctgccatcattgcaaagattgctgactacggcattgctcagtactg

SEQ ID NO: 2 - DNA, 57 bp, Artificial sequence
  partial sequence of mutant LRRK2
gtatcccaatgctgccatcattgcaaagattgctgactacagcattgctcagtactg

SEQ ID NO: 3 - DNA, 20 bp, Artificial sequence
  gRNA
taacgtttctaacgactgat

SEQ ID NO: 4 - DNA, 23 bp, Artificial sequence
  gRNA
cagcattgctcagtactgctgta

SEQ ID NO: 5 - DNA, 23 bp, Artificial sequence
  gRNA
actacagcattgctcagtactgc

SEQ ID NO: 6 - DNA, 23 bp, Artificial sequence
  gRNA
tctaacgactgatgtcgtaacga

SEQ ID NO: 7 - DNA, 23 bp, Artificial sequence
  gRNA
atgtcgtaacgagtcatgacgac

SEQ ID NO: 8 - DNA, 7584 bp, Artificial sequence
  wild type LRRK2
atggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 9 - protein, 2527 aa, Artificial sequence
  WT LRRK2 protein
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 10 - protein, 9 aa, Artificial sequence
  meganuclease motif
LAGLIDADG

SEQ ID NO: 11 - DNA, 20 bp, Artificial sequence
  gRNA
gcctggagggaaagacacaa

SEQ ID NO: 12 - DNA, 49 bp, Artificial sequence
  gRNA
caaaggaagttttccaggcatctgtgaatgcattgtcaactctcttaga

SEQ ID NO: 13 - DNA, 20 bp, Artificial sequence
  gRNA
tagtcagcaatctttgcaat

SEQ ID NO: 14 - DNA, 20 bp, Artificial sequence
  gRNA
gtgactagaaataaaatatc

SEQ ID NO: 15 - DNA, 20 bp, Artificial sequence
  gRNA
tgactagaaataaaatatca

SEQ ID NO: 16 - DNA, 20 bp, Artificial sequence
  gRNA
gaatcacaggggaagaagaa

SEQ ID NO: 17 - DNA, 20 bp, Artificial sequence
  gRNA
gaaactggagcagctcattt

SEQ ID NO: 18 - DNA, 20 bp, Artificial sequence
  gRNA
cagaacaaatcacctttata

SEQ ID NO: 19 - DNA, 20 bp, Artificial sequence
  gRNA
tgtgttgaataatgatgagt

SEQ ID NO: 20 - DNA, 20 bp, Artificial sequence
  gRNA
gaataatgataagttggaat

SEQ ID NO: 21 - DNA, 20 bp, Artificial sequence
  gRNA
atcattattcaacataatat

SEQ ID NO: 22 - DNA, 49 bp, Artificial sequence
  LRRK2 gRNA target nucleic acid sequence
gttttgtgtctttccctccaggctcgcgcttcttcttcccctgtgattc

SEQ ID NO: 23 - DNA, 49 bp, Artificial sequence
  LRRK2 gRNA target nucleic acid sequence
gttttgtgtctttccctccaggctagcgcttcttcttcccctgtgattc

SEQ ID NO: 24 - DNA, 49 bp, Artificial sequence
  LRRK2 gRNA target nucleic acid sequence
caaaggaagttttccaggcatctgcgaatgcattgtcaactctcttaga

SEQ ID NO: 25 - DNA, 49 bp, Artificial sequence; identical to SEQ ID NO: 12
  LRRK2 gRNA target nucleic acid sequence

SEQ ID NO: 26 - DNA, 49 bp, Artificial sequence
  LRRK2 gRNA target nucleic acid sequence
atcattgcaaagattgctgactacggcattgctcagtactgctgtagaa

SEQ ID NO: 27 - DNA, 49 bp, Artificial sequence
  LRRK2 gRNA target nucleic acid sequence
atcattgcaaagattgctgactacagcattgctcagtactgctgtagaa

SEQ ID NO: 28 - DNA, 49 bp, Artificial sequence
  LRRK2 gRNA target nucleic acid sequence
gtgactagaaataaaatatcagggatatgctcccccttgagactgaagg

SEQ ID NO: 29 - DNA, 49 bp, Artificial sequence
  LRRK2 gRNA target nucleic acid sequence
gtgactagaaataaaatatcaggggtatgctcccccttgagactgaagg

SEQ ID NO: 30 - DNA, 49 bp, Artificial sequence
  LRRK2 gRNA target nucleic acid sequence
ttttgtgtctttccctccaggctcgcgcttcttcttcccctgtgattct

SEQ ID NO: 31 - DNA, 49 bp, Artificial sequence
  LRRK2 gRNA target nucleic acid sequence
ttttgtgtctttccctccaggctcacgcttcttcttcccctgtgattct

SEQ ID NO: 32 - DNA, 49 bp, Artificial sequence
  LRRK2 gRNA target nucleic acid sequence
agagaaactggagcagctcattttagaagggtaagaaagagctcattaa

SEQ ID NO: 33 - DNA, 49 bp, Artificial sequence
  LRRK2 gRNA target nucleic acid sequence
agagaaactggagcagctcattttggaagggtaagaaagagctcattaa

SEQ ID NO: 34 - DNA, 49 bp, Artificial sequence
  LRRK2 gRNA target nucleic acid sequence
gatgccatgaagccttggctcttcaatataaaggtgatttgttctgatc

SEQ ID NO: 35 - DNA, 49 bp, Artificial sequence
  LRRK2 gRNA target nucleic acid sequence
gatgccatgaagccttggctcttccatataaaggtgatttgttctgatc

SEQ ID NO: 36 - DNA, 49 bp, Artificial sequence
  LRRK2 gRNA target nucleic acid sequence
ttggctgacctgcctagaaatattatgttgaataatgatgagttggaat

SEQ ID NO: 37 - DNA, 49 bp, Artificial sequence
  LRRK2 gRNA target nucleic acid sequence
ttggctgacctgcctagaaatattgtgttgaataatgatgagttggaat

SEQ ID NO: 38 - DNA, 49 bp, Artificial sequence
  LRRK2 gRNA target nucleic acid sequence
agaaatattatgttgaataatgatgagttggaatttgaacaagctccag

SEQ ID NO: 39 - DNA, 49 bp, Artificial sequence
  LRRK2 gRNA target nucleic acid sequence
agaaatattatgttgaataatgataagttggaatttgaacaagctccag

SEQ ID NO: 40 - DNA, 3900 bp, Artificial sequence
  An exemplary DNA sequence of Cpf1
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 41 - protein, 1345 aa, Artificial sequence
  An exemplary amino acid sequence of Cpf1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNKRPAATKKAGQAKKKKGSYPYDVPDYAYPYDVPDYAYPYDVPDYA